(19) United States
(12) Patent Application Publication
(10) Pub. No.: US 2025/0134354 A1
(43) Pub. Date: May 1, 2025

(54) DISTAL TIPS FOR MEDICAL DEVICES

(71) Applicants: Boston Scientific Scimed, Inc., Maple Grove, MN (US); Boston Scientific Medical Device Limited, Galway (IE)

(72) Inventors: Aditya DHANOTIYA, Indore (IN); Shrikant Vasant RAUT, Mumbai (IN); Pooja Bhuvanesh KULKARNI, Pune (IN); James WELDON, Newton, MA (US)

(73) Assignees: Boston Scientific Scimed, Inc., Maple Grove, MN (US); Boston Scientific Medical Device Limited, Galway (IE)

(21) Appl. No.: 18/922,648

(22) Filed: Oct. 22, 2024

Related U.S. Application Data
(60) Provisional application No. 63/592,938, filed on Oct. 25, 2023.

(51) Int. Cl. A61B 1/00; A61B 1/05

(52) U.S. Cl. CPC A61B 1/00096; A61B 1/05

(57) ABSTRACT

A distal tip of a duodenoscope may comprise: a first side surface; a second side surface; a top surface; a bottom surface; and a distal surface. The first side surface, the top surface, and the distal surface meet at a first junction. The first side surface, the bottom surface, and the distal surface may meet at a second junction. The second side surface, the bottom surface, and the distal surface may meet at a third junction. The second side surface, the top surface, and the distal surface may meet at a fourth junction. The first junction and the fourth junction may be approximately symmetrical. The second junction and the third junction may be approximately symmetrical. The first junction and the third junction may be approximately symmetrical. The second junction and the fourth junction may be approximately symmetrical.

### CROSS-REFERENCE TO RELATED APPLICATIONS

This application claims the benefit of priority under 35 U.S.C. § 119 from U.S. Provisional Application No. 63/592,938, filed Oct. 25, 2023, which is incorporated by reference herein in its entirety.

### TECHNICAL FIELD

Various aspects of this disclosure relate generally to distal tips for medical devices. In particular, aspects of this disclosure pertain to distal tips for duodenoscopes, among other aspects.

### BACKGROUND

Duodenoscopes may include a handle and a sheath, and the sheath may be insertable into a body lumen of a subject. The sheath may terminate in a distal tip portion, which may include features such as optical elements (e.g., camera, lighting, etc.), air/water outlets, and working channel openings. An elevator may be disposed at a distal tip and may be actuatable in order to change an orientation of a medical device/tool passed through the working channel. For example, the elevator may be pivotable or otherwise movable.

During an endoscopic retrograde cholangiopancreatography (ERCP) procedure, an operator may insert a duodenoscope into a body lumen and navigate the distal tip to a procedure site. One potential risk associated with ERCP is a risk of perforation, in which the distal tip may perforate a wall of the body lumen (e.g., a wall of the esophagus or intestine). Such perforations are a serious complication with a high mortality rate. Therefore, a need exists for distal tips of medical devices that reduce a risk of complications.

### SUMMARY

Aspects of the disclosure relate to distal tips of endoscopes that decrease a force necessary to advance the tip or otherwise have a streamlined profile. Each of the aspects disclosed herein may include one or more of the features described in connection with any of the other disclosed aspects.

In an example, a distal tip of a duodenoscope may comprise: a first side surface; a second side surface; a top surface; a bottom surface; and a distal surface. The first side surface, the top surface, and the distal surface meet at a first junction. The first side surface, the bottom surface, and the distal surface may meet at a second junction. The second side surface, the bottom surface, and the distal surface may meet at a third junction. The second side surface, the top surface, and the distal surface may meet at a fourth junction. The first junction and the fourth junction may be approximately symmetrical. The second junction and the third junction may be approximately symmetrical. The first junction and the third junction may be approximately symmetrical. The second junction and the fourth junction may be approximately symmetrical.

Any of the aspects disclosed herein may include any of the following features, alone or in any combination. Upon viewing the distal surface of the distal tip facing proximally, the distal surface may be approximately symmetrical about at least two planes including a line that extends parallel to or coaxial with a central longitudinal axis of the distal tip. A portion of the distal surface may be approximately symmetrical about at least four planes including a line that extends parallel to or coaxial with a central longitudinal axis of the distal tip. The first side surface and the second side surface may flare outwardly in a distal direction. Each of the first side surface and the second side surface may include a proximal portion, a distal portion, and a middle portion. The middle portion may be inward, closer to the central longitudinal axis, of the distal portion and the proximal portion. The top surface may define a first opening and a second opening. The first opening may be configured to receive an imaging device, and wherein the second opening is configured to receive a lighting element. The top surface may include a cover that spans a space between the first opening and the second opening. The cover may be integrally formed with a remainder of the top surface. The top surface may include at least one opening for receiving an imaging device or a lighting element, and wherein a first portion of the top surface that is distal to the at least one opening tapers downward toward the distal surface. The first portion may curve in a lateral direction. The at least one opening may be at least one first opening. The top surface further defines a second opening for receiving an elevator, and wherein a side of the first portion that is closer to the second opening is higher than a side of the first portion that is further from the second opening. At least a portion of the distal tip may have a cross-sectional shape perpendicular to the central longitudinal axis that is approximately circular. The top surface may taper downward from a proximal end of the top surface to a distal end of the top surface. A central portion of the distal surface may extend further in a distal direction than side portions of the distal surface do.

In another aspect, a distal tip of a duodenoscope may comprise: a first side surface; a second side surface; a top surface; a bottom surface; and a distal surface. Each of the first side surface and the second side surface may include a proximal portion, a distal portion, and a middle portion. The middle portion may be inward of the respective distal portion and the respective proximal portion.

Any of the aspects disclosed herein may have any of the following features alone or in any combination. The bottom surface may include a proximal portion, a distal portion, and a middle portion. The middle portion may be higher than the proximal portion and the distal portion.

In a further aspect, a distal tip of a duodenoscope may comprise: a first side surface; a second side surface; a top surface; a bottom surface; and a distal surface. The top surface may include at least one opening for receiving an imaging device or a lighting source. A first portion of the top surface that is distal to the at least one opening tapers downward toward the distal surface and curves in a lateral direction.

Any of the aspects disclosed herein may have any of the following features. The at least one opening may be at least one first opening. The top surface may further define a second opening for receiving an elevator. A side of the first portion that is closer to the second opening may be higher than a side of the first portion that is further from the second opening. A distal portion of the second opening may include a recess defined by a rim. The rim may include an apex and two tapered side portions. A first of the two tapered side portions may be continuous with an edge of the first portion.

### BRIEF DESCRIPTION OF THE DRAWINGS

The accompanying drawings, which are incorporated in and constitute a part of this specification, illustrate aspects this disclosure and together with the description, serve to explain the principles of the disclosure.

FIGS. 1A and 1B depict an exemplary medical device.

FIGS. 2A-2E depict aspects of an exemplary distal tip of the medical device of FIGS. 1A and 1B.

FIGS. 3A-3D depict aspects of another exemplary distal tip of the medical device of FIGS. 1A and 1B.

FIGS. 4A-4G depict aspects of a further exemplary distal tip of the medical device of FIGS. 1A and 1B.

FIGS. 5A-5D depict aspects of another exemplary distal tip of the medical device of FIGS. 1A and 1B.

FIGS. 6A-6D depict aspects of an additional exemplary distal tip of the medical device of FIGS. 1A and 1B.

FIGS. 7A-7D depict aspects of a further exemplary distal tip of the medical device of FIGS. 1A and 1B.

FIGS. 8A-8E depict aspects of yet another exemplary distal tip of the medical device of FIGS. 1A and 1B.

### DETAILED DESCRIPTION

Distal tips of medical devices, such as duodenoscopes, may pose risks of perforating through a wall of a body lumen of a subject. The disclosed distal tips include features that help decrease a risk of such perforations. For example, as compared with conventional distal tips the disclosed distal tips may have increased curvature of their surfaces, increased symmetry, or other features to decrease an amount of force required to insert advance the distal tip and/or to prevent damage to tissue. For example, the disclosed distal tips may lack sharp edges or may have softer forms. Furthermore, the disclosed distal tips may reduce areas that are prone to build up of pathogens (e.g., bacteria). The disclosed distal tips may also include more space to house elements of the distal tip (e.g., electronics, such as imaging devices and/or lighting elements). The disclosed distal tips may direct the scope. The disclosed distal tips may further increase a range of motion of an elevator of the distal tip.

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. In some drawings, arrows labeled “P” and “D” indicate proximal and distal directions, respectively. The term “exemplary” is used in the sense of “example,” rather than “ideal.” The term “approximately,” or like terms (e.g., “substantially”), includes values +/−10% of a stated value.

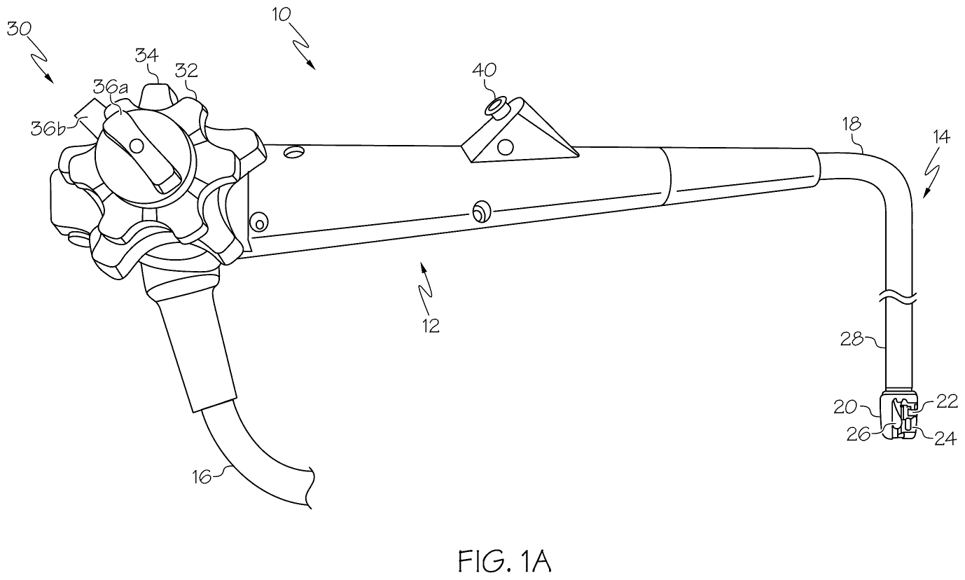

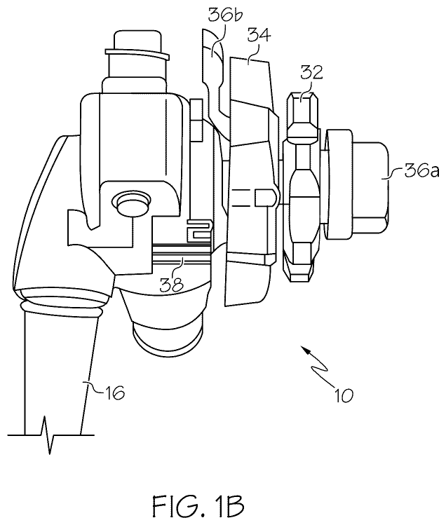

FIG. 1A depicts an exemplary medical device 10 having a handle 12 and an insertion portion 14. FIG. 1B shows a proximal end of handle 12. Medical device 10 may also include an umbilicus 16 for purposes of connecting medical device 10 to sources of, for example, air, water, suction, power, etc., as well as to image processing and/or viewing equipment. Although duodenoscopes and endoscopes (and combination devices that perform functions of duodenoscopes and endoscopes) are particularly referenced herein, the disclosure also encompasses other types of devices, such as bronchoscopes, gastroscopes, endoscopic ultrasound (“EUS”) scopes, colonoscopes, ureteroscopes, bronchoscopes, laparoscopes, cystoscopes, aspiration scopes, sheaths, catheters, or similar devices. A reference to a duodenoscope herein should be understood to encompass any of the above medical devices.

Insertion portion 14 may include a sheath or shaft 18 and a distal tip 20. Distal tip 20 may include an imaging device 22 (e.g., a camera) and a lighting element 24 (e.g., a light emitting diode (LED) or an optical fiber). Although the term “lighting element” is used herein, it will be appreciated that the term “lighting element” may include a plurality of lighting elements (e.g., a plurality of LEDs or optical fibers). Distal tip 20 may be side-facing. That is, imaging device 22 and lighting element 24 may face radially outward, perpendicularly, approximately perpendicularly, or otherwise transverse to a longitudinal axis of shaft 18 and distal tip 20. However, the disclosure is not limited to such an arrangement.

Distal tip 20 may also include an elevator 26 for changing an orientation of an accessory device or a tool inserted in a working channel of medical device 10. Elevator 26 may alternatively be referred to as a swing stand, pivot stand, raising base, or any suitable other term. Elevator 26 may be pivotable via, e.g., an actuation wire or another control element that extends from handle 12, through shaft 18, to elevator 26.

A distal portion of shaft 18 that is connected to distal tip 20 may have a steerable section 28. Steerable section 28 may be, for example, an articulation joint. Shaft 18 and steerable section 28 may include a variety of structures that are known or may become known in the art.

Handle 12 may have one or more actuators/control mechanisms 30. One or more of control mechanisms 30 may provide control over steerable section 28. One or more of control mechanisms 30 may allow for provision of air, water, suction, etc. For example, handle 12 may include control knobs 32, 34 for left, right, up, and/or down control of steerable section 28. For example, one of knobs 32, 34 may provide left/right control of steerable section 28, and the other of knobs 32, 34 may provide up/down control of steerable section 28. Handle 12 may further include one or more locking mechanisms 36a, 36b (e.g., knobs or levers) for preventing steering and/or braking of steerable section 28 in at least one of an up, down, left, or right direction. Handle 12 may include an elevator control lever 38 (see FIG. 1B). Elevator control lever 38 may raise and/or lower elevator 26, via connection between lever 38 and an actuating wire that extends from lever 38, through shaft 18, to elevator 26. A port 40 may allow passage of a tool through port 40, into a working channel of the medical device 10, through shaft 18, to distal tip 20.

In use, an operator may insert at least a portion of shaft 18 into a body lumen of a subject. Distal tip 20 may be navigated to a procedure site in the body lumen. For example, the operator may push distal tip 20 distally in order to advance it through the body lumen. The operator may insert an accessory device (not shown) into port 40, and pass the accessory device through shaft 18 via a working channel to distal tip 20. The accessory device may exit the working channel at distal tip 20. The user may use elevator control lever 38 to raise elevator 26 and angle the accessory device toward a desired location (e.g., a papilla of the pancreatico-biliary tract). The user may use the accessory device to perform a medical procedure.

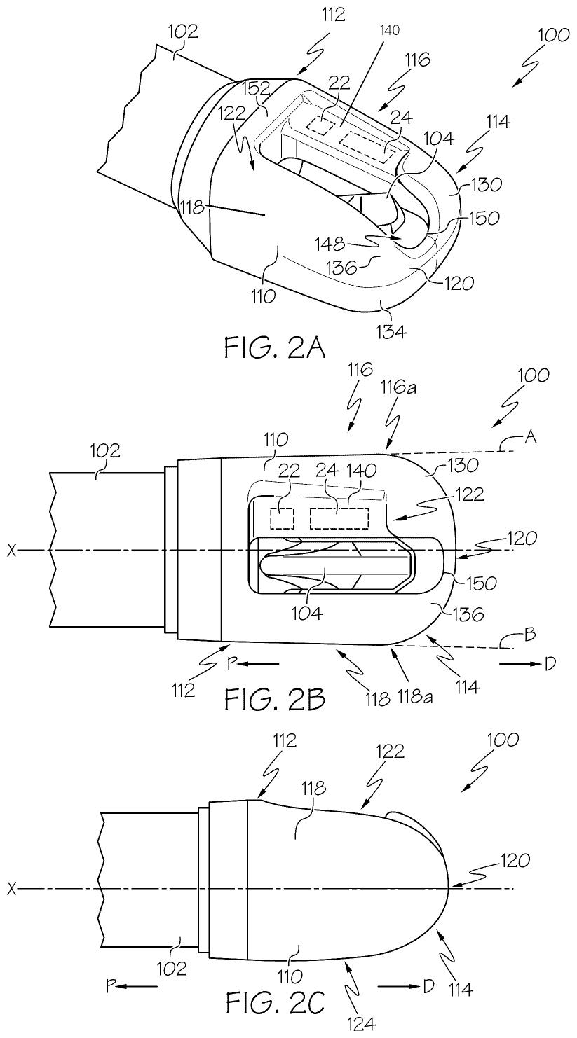

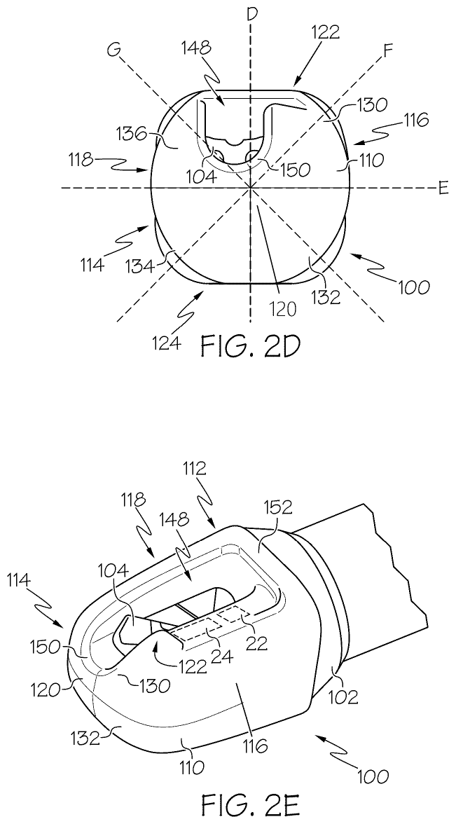

FIGS. 2A-2E depict a first exemplary distal tip 100, which may be used in conjunction with medical device 10 in place of distal tip 20. Distal tip 100 may have any of the features of distal tip 20. Distal tip 100 may include a core 102 and a cap 110. Core 102 may be inserted into cap 110 during assembly of distal tip 100. Core 102 may have mounted thereon or, otherwise attached thereto, elements such as imaging device 22 and lighting element 24, which are shown mounted with a potting material 140. Distal tip 100 may also include an elevator 104, having any of the properties of elevator 26. Elevator 104 may be rotatable/pivotable with respect to other portions of distal tip 100 in order to raise and/or lower an accessory device or tool passed through a working channel of shaft 18. Further details of distal tip 100 that accommodate elevator 104 will be described below. In alternatives, core 102 may be omitted or core 102 and cap 110 may be a single, unitary structure.

Cap 110 of distal tip 100 may extend between a proximal end 112 and a distal end 114. A distal surface 120 may be disposed at distal end 114. Cap 110 may have a first side surface 116 and a second side surface 118. First side surface 116 may be on an opposite side of cap 110 from second side surface 118. First side surface 116 and second side surface 118 may be lateral side surfaces of cap 110.

Cap 110 may have a top surface 122 and a bottom surface 124. Top surface 122 may be on an opposite side of cap 110 from bottom surface 124. Top surface 122 of cap 110 may be on a side/portion of cap 110 that includes surfaces or openings for receiving electronics (e.g., imaging device 22 and lighting element 24). At least a portion of top surface 122 that is adjacent to second side surface 118 may taper downward (toward bottom surface 124), as shown in FIG. 2C. Top surface 122 may also include an opening 148 to accommodate elevator 104. As elevator 104 rotates/pivots, a range of motion of an accessory device or tool (not shown) may pass through various portions of opening 148.

Distal end 114 may include a recess 150 that is in communication with opening 148. An accessory device extending along elevator 104, when elevator 104 is in a lowered configuration (as shown in the Figures), may extend through (and may optionally contact) recess 150. As shown particularly in FIG. 2A, recess 150 may have rounded, smooth surfaces (a rounded/smooth rim). Although a proximal portion 152 of cap 110 is shown as lacking a protrusion for interacting with elevator 104 when it is in a raised configuration, this disclosure is not so limited. For example, it will be appreciated that proximal portion 152 (FIGS. 2A and 2E) or another portion of distal tip 100 (e.g., core 102) may include such a feature for locking or otherwise interacting with elevator 104.

Generally, intersections between distal surface 120, first side surface 116, second side surface 118, top surface 122, and bottom surface 124 may be rounded without sharp corners formed at junctions between the various surfaces. Such rounded junctions may provide for smoother interactions between distal tip 100 and surfaces of a subject's body lumen. Distal tip 100 may also have a streamlined shape, such that advancing distal tip 100 (e.g., distally) requires relatively less force than advancing a conventional distal tip.

First side surface 116, top surface 122, and distal surface 120 may meet at a first junction 130. First side surface 116, bottom surface 124, and distal surface 120 may meet at a second junction 132. Second side surface 118, bottom surface 124, and distal surface 120 may meet at a third junction 134. Second side surface 118, top surface 122, and distal surface 120 may meet at a fourth junction 136.

Junctions 130, 132, 134, 136 may be approximately symmetrical, as shown in FIG. 2D. Symmetry, discussed below, is discussed as if opening 148/recess 150 did not exist. For example, except for junctions 130, 132, 134, 136 and recess 150/opening 148, distal surface 120 may be approximately symmetrical about a plane D that extends parallel to or coaxial with a central longitudinal axis X (labeled in FIGS. 2B and 2C) of distal tip 100 and/or cap 110 and between top surface 122 and bottom surface 124. Distal surface 120 may be approximately symmetrical about plane E (FIG. 2D) that extends parallel to or coaxial with central longitudinal axis X of distal tip 100 and/or cap 110 and between top first side surface 116 and second side surface 118. Distal surface 120 may be approximately symmetrical about plane F (FIG. 2D) that extends parallel to or coaxial with central longitudinal axis X of distal tip 100 and/or cap 110 and between first junction 130 and third junction 134. Distal surface 120 may be approximately symmetrical about plane G (FIG. 2D) that extends parallel to or coaxial with a central longitudinal axis X of distal tip 100 and/or cap 110 and between second junction 132 and fourth junction 136. Thus, a portion of distal surface 120 may be symmetrical about six planes or at least four planes.

In other words, junctions 130, 132, 134, 136 may have similar or the same shapes but may be rotated about central longitudinal axis X of cap 110. Junctions 130, 132, 134, 136 may all be filleted to form rounded, smooth interfaces between the relevant surfaces. Distal end 114/distal surface 120 may be a blunt, rounded end, which may transition to side surfaces 116, 118, top surface 122, and bottom surface 124 via junctions 130, 132, 134, 136. As shown particular in FIG. 2D, when viewing distal end 114 facing a proximal direction, distal end 114 may have an approximately circular shape, which may correspond to a shape of a cross-section of distal end 114 perpendicular to central longitudinal axis X. Similarly, as shown in FIG. 2C, when viewing distal tip 100 from the side, viewing side surface 118, distal end 114 may have an approximately semicircular, rounded shape (when not including opening 148/recess 150). When viewing distal surface 120 from above (FIG. 2B), a central portion of distal surface 120 may extend further distally than side portions of distal surface 120 extend distally. In other words, side portions of distal surface 120 may be proximal of a central portion of distal surface 120. Overall, distal end 114 may have a shape that is similar to (i.e., approximately) a semi-sphere.

As shown particularly in FIG. 2B, first side surface 116 and second side surface 118 may flare laterally outward in a distal direction. In other words, a distance between first side surface 116 and second side surface 118 may be smaller at more proximal portions of side surfaces 116, 118 than at more distal portions of side surfaces 116, 116. As shown in FIG. 2B, lines A and B drawn alongside surfaces 116, 118, respectively, may diverge from one another/be angled laterally outward in the distal direction. Such a shape of side surfaces 116, 118 may allow for improved navigation of distal tip 100. As distal tip 100 moves through a body lumen, distal ends 116a, 118a of side surfaces 116, 118, respectively, may contact and walls of a body lumen. More contact may be made with distal surface 120 and junctions 130, 132, 134, 136 than proximal portions of side surfaces 116, 118. In some examples, proximal portions of side surfaces 116, 118 may not contact a wall of a body lumen. Because more proximal portions of distal tip 100 (proximally of distal ends 116a, 116a) may be relatively narrower, portions of distal tip 100 that are proximal of distal ends 116a, 118a may move easily through the body lumen.

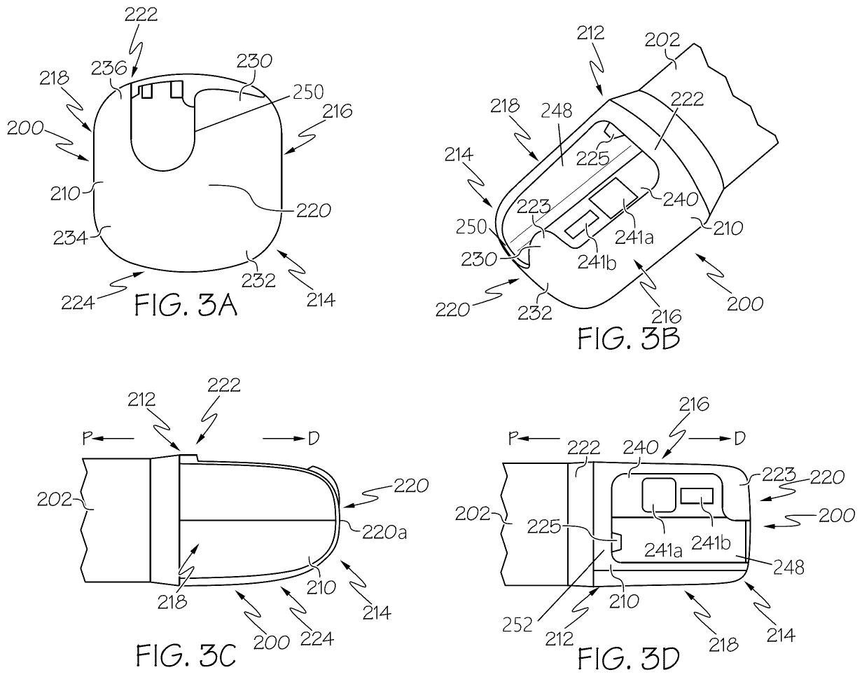

FIGS. 3A-3D depict an alternative distal tip 200, having any of the features of distal tip 100, unless otherwise specified herein. For sake of clarity, differences between distal tip 200 and distal tip 100 may be highlighted herein, while similarities may be omitted. Distal tip 200 may be used in conjunction with medical device 10 in place of distal tip 20.

Distal tip 200 may include a core 202 and a cap 210. Core 202 may be inserted into cap 210 during assembly of distal tip 200. Core 202 may have mounted thereon or, otherwise attached thereto, elements such as imaging device 22 and lighting element 24. Distal tip 200 may also include an elevator (not shown), having any of the properties of elevators 26, 104.

Cap 210 of distal tip 200 may extend between a proximal end 212 and a distal end 214. A distal surface 220 may be disposed at distal end 214. Cap 210 may have a first side surface 216 and a second side surface 218. First side surface 216 may be on an opposite side of cap 210 from second side surface 218. First side surface 216 and second side surface 218 may be lateral side surfaces of cap 210. Cap 210 may have a top surface 222 and a bottom surface 224. Top surface 222 may be on an opposite side of cap 210 from bottom surface 224. Top surface 222 of cap 210 may be a side of cap 210 that includes surfaces or openings for receiving electronics (e.g., imaging device 22 and lighting element 24).

Top surface 222 may include an opening 248 for accommodating an elevator, similar to opening 148, described above. Distal surface 220 may include a recess 250 (FIG. 3A), similar to recess 150. As compared with recess 150, recess 250 may be less slanted in a proximal/distal direction and may extend more along an up/down direction of FIG. 3A. The shape of recess 150 may be due to distal surface 220 having a more planar shape than distal surface 120 and/or due to top surface 222 extending more levelly than top surface 122, as described in further detail below. A proximal portion 252 of top surface 222 may include a boss 225 for interacting with and, in some examples, locking the elevator.

Generally, intersections between distal surface 220, first side surface 216, second side surface 218, top surface 222, and bottom surface 224 may be rounded without sharp corners formed at junctions between the various surfaces. Such rounded junctions may provide for smoother interactions between distal tip 200 and surfaces of a subject's body lumen. Distal tip 200 may also have a streamlined shape, such that advancing distal tip 200 (e.g., distally) requires relatively less force than advancing a conventional distal tip.

First side surface 216, top surface 222, and distal surface 220 may meet at a junction 230. First side surface 216, bottom surface 224, and distal surface 220 may meet at a junction 232. Second side surface 218, bottom surface 224, and distal surface 220 may meet at a junction 234. Second side surface 218, top surface 222, and distal surface 220 may meet at a junction 236. Junctions 230, 232, 234, 236 may be rounded such that, when viewing distal end 214 facing proximally (as shown in FIG. 3A), distal end 214 may have a rounded square shape. When viewing distal end 214 facing proximally, side surfaces 216, 218 may be approximately straight, in an up/down direction. Bottom surface 224 may be convex shaped with a large radius of curvature (i.e., gently curved) when viewing distal end 214 facing proximally. A portion 221 of top surface 222 that is proximal of an opening 248 (discussed below) may similarly be convex shaped with a large radius of curvature (i.e., gently curved) when viewing distal end 214 facing proximally.

As with distal tip 100, the discussions of symmetry below are discussed as if opening 248/recess 250 are not present. Lines showing axes/planes of distal tip 200 are omitted in FIGS. 3A-3D for clarity of illustration but may be similar to the lines depicted in FIGS. 2A-2E and discussed above. Distal end 214 may be symmetrical about a plane (similar to plane E) that extends along a central longitudinal axis of distal tip 200 (similar to central longitudinal axis X of distal tip 100) and extends from first side surface 216 to second side surface 218. Distal end 214 may be symmetrical about a plane (similar to plane D) that extends along the central longitudinal axis of distal tip 200 and extends from top surface 222 to bottom surface 224. In other words, distal end 214 may be symmetrical about a plane that extends through a central longitudinal axis of cap 210 and extends up/down in FIG. 3A and also symmetrical about a plane that extends through a central longitudinal axis of cap 210 and extends left/right in FIG. 3A. Junctions 230, 232, 234, 236 may be symmetrical with one another. In other words, junctions 230, 232, 234, 236 may be approximately symmetrical, as shown in FIG. 3D. In other words, junctions 230, 232, 234, 236 may have similar or the same shapes but may be rotated about a central longitudinal axis of cap 210.

As shown in FIG. 3C, distal surface 220 may have a planar portion 220a. Planar portion 220a may extend approximately perpendicularly to a central longitudinal axis of cap 210. Junctions 230, 232, 234, 236 may form a curved transition between planar portion 220a, top surface 222, and bottom surface 224. A radius of curvature of junctions 230, 232, 234, 236 may be large so as to help provide better sliding/gliding through a body lumen of a subject.

As compared with junctions 130, 132, 134, 136, junctions 230, 232, 234, 236 may curve less gradually downward/upward to distal surface 220. In other words, a radius of curvature to transition between top surface 222 and distal surface 220, as well as bottom surface 224 and distal surface 220 may be smaller than for corresponding elements of distal tip 100. The shape of junctions 230, 232, 234, 236 may contribute to distal surface 220 having planar portion 220a.

Distal tip 200 may include a cover 240, which may extend over a well for mounting imaging device 22 and lighting element 24. Cover 240 may have openings 241a, 241b formed therein. Cover 240 may extend between openings 241a, 241b to cover the space therebetween. Imaging device 22 and lighting element 24 may be mounted within distal tip 200 so that imaging device 22 aligns with opening 241a and lighting element 24 aligns with opening 241b, or vice versa. Although cover 240 is depicted as having two openings 241a, 241b, it will be appreciated that cover 240 may include any suitable number of openings, corresponding to a number of electronic devices that cover 240 encloses/shields/covers. Cover 240 may be integrally formed with cap 210 (including a remainder of top surface 222), as a single piece of material. Alternatively, cover 240 may be a separate piece of material. Cover 240 may help to reduce a presence of sharp edges associated with imaging device 22 and lighting element 24. Cover 240 may be recessed with respect to surrounding portions of top surface 222. There may be a smooth transition between cover 240 and top surface 222, such that hard edges are avoided therebetween. A portion 223 of top surface 222 that is distal to cover 240 may be curved, forming a smooth transition with distal surface 220.

Comparing distal tip 100 to distal tip 200, top surface 122 of distal tip 100 may taper downward, as discussed above, while top surface 122 may be approximately level (approximately parallel to a central longitudinal axis of distal tip 200). Whereas distal surface 120 of distal tip 100 may have an approximately circular shape when viewing distal surface 120 facing a proximal direction, distal surface 220 may have a rounded square shape. While side surfaces 116, 118 may flare outward, side surfaces 216, 218 may be approximately parallel to one another and/or a central longitudinal axis of distal tip 200. Distal tip 100 may lack a structure similar to cover 240. The differences above are not exclusive, and other differences between distal tips 100, 200 exist. Furthermore, it will be appreciated that features of distal tip 100 may be combined with features of distal tip 200 in any suitable arrangement. For example, distal tip 200 may have flared side surfaces 216, 218. Distal tip 100 may include a cover similar to cover 240.

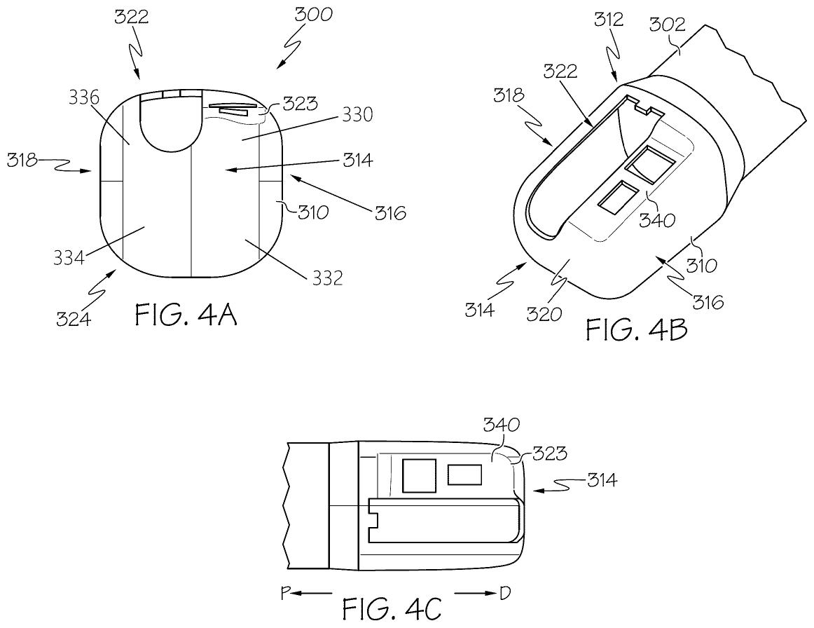

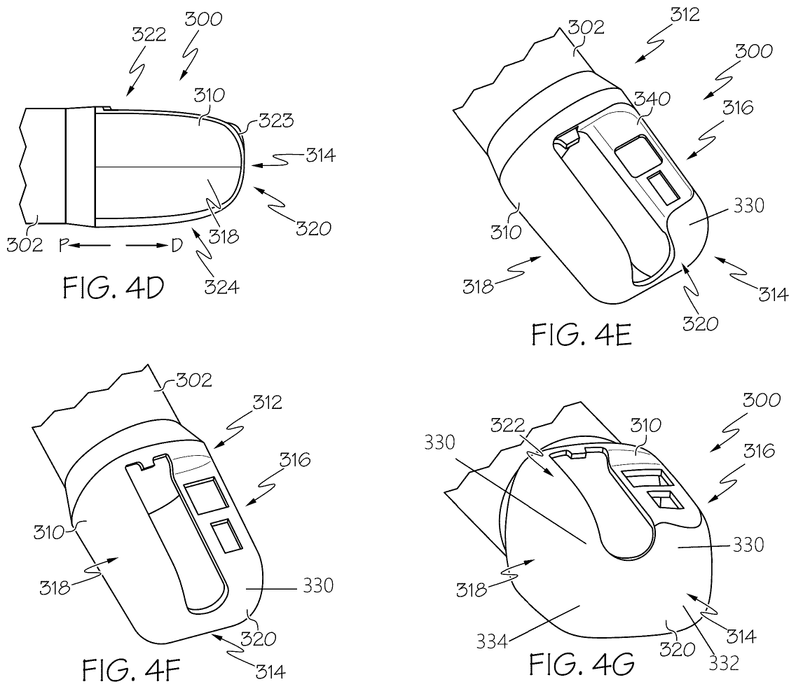

FIGS. 4A-4G depict an alternative distal tip 300, having any of the features of distal tips 100, 200 unless otherwise specified herein. For sake of clarity, differences between distal tip 300 and distal tips 100, 200 may be highlighted herein, while similarities may be omitted. In particular, distal tip 300 may be similar to distal tip 200. Distal tip 300 may be used in conjunction with medical device 10 in place of distal tip 20.

Distal tip 300 may include a core 302 and a cap 310. Core 302 may be inserted into cap 310 during assembly of distal tip 300. Core 302 may have mounted thereon or, otherwise attached thereto, elements such as imaging device 22 and lighting element 24. Distal tip 300 may also include an elevator (not shown), having any of the properties of elevators 26, 104.

Cap 310 of distal tip 300 may extend between a proximal end 312 and a distal end 314. A distal surface 320 may be disposed at distal end 314. Cap 310 may have a first side surface 316 and a second side surface 318. First side surface 316 may be on an opposite side of cap 310 from second side surface 318. First side surface 316 and second side surface 318 may be lateral side surfaces of cap 310. Cap 310 may have a top surface 322 and a bottom surface 324. Top surface 322 may be on an opposite side of cap 310 from bottom surface 324. Top surface 322 of cap 310 may be a side of cap 310 that includes surfaces or openings for receiving electronics (e.g., imaging device 22 and lighting element 24).

Generally, intersections between distal surface 320, first side surface 216, second side surface 318, top surface 322, and bottom surface 324 may be rounded without sharp corners formed at junctions between the various surfaces. Such rounded junctions may help provide for smoother interactions between distal tip 300 and surfaces of a subject's body lumen. Distal tip 300 may also have a streamlined shape, such that advancing distal tip 300 (e.g., distally) requires relatively less force than advancing a conventional distal tip.

First side surface 316, top surface 322, and distal surface 320 may meet at a junction 330. First side surface 316, bottom surface 324, and distal surface 320 may meet at a junction 332. Second side surface 318, bottom surface 324, and distal surface 320 may meet at a junction 334. Second side surface 318, top surface 322, and distal surface 320 may meet at a junction 336.

Similarly to top surface 222, top surface 322 may include a cover 340, having any of the properties of cover 240. A portion 323 of top surface 322 that is distal to cover 340 and/or cover 340 itself may be blended or at least partially integrated with other surfaces of cap 310. Although shapes of portion 323 are discussed below, it will be appreciated that the same features may alternatively apply to cover 340. With particular reference to FIGS. 4A and 4G, portion 323 may taper downward (toward bottom surface 324) along a direction toward first side surface 316. In other words, portion 323 may taper downward in a laterally outward direction. The taper may be curved (e.g., wave shaped) to provide for smooth blending between top surface 322 and first side surface 316. In some examples, an entirety of cover 340 may taper downward in a laterally outward direction (a direction toward first side surface 316). As shown in FIGS. 4A and 4G, proximal portions of cover 340 may be higher (further from bottom surface 324) than distal portions of cover 340 and or portion 323.

Comparing distal tip 200 to distal tip 300, particular reference is made to FIGS. 3A, 3C, 4A, and 4D. As shown in FIG. 3C (viewing distal tip 200 from the side, facing second surface 218) portion 223 of top surface 222 and/or junction 230 may extend slightly distally of side junction 236 and an adjacent portion of top surface 222. Portion 233 may protrude slightly. In contrast, as shown in FIG. 4D (viewing distal tip 300 from the side, facing second side surface 318), portion 323 may blend smoothly with junction 330 and may not so protrude/extend further distally. Comparing FIG. 3A (distal tip 200) and 4A (distal tip 300), portion 223 may extend approximately parallel to a central longitudinal axis of distal tip 200, while portion 323 may taper downward and blend into junction 330/distal surface 320 of distal tip 300.

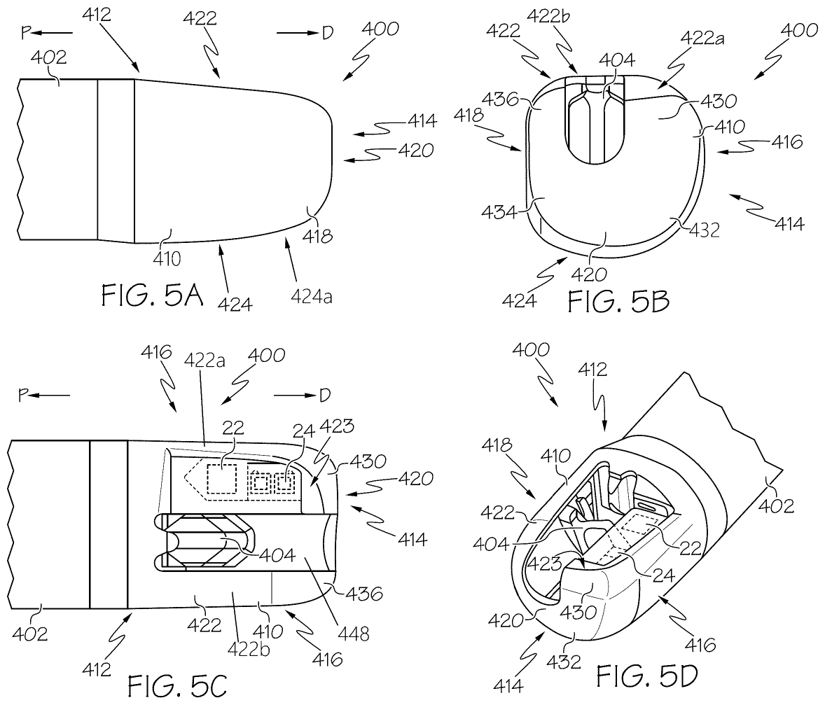

FIGS. 5A-5D depict an alternative distal tip 400, having any of the features of distal tips 100, 200, 300 unless otherwise specified herein. For sake of clarity, differences between distal tip 400 and distal tips 100, 200, 300 may be highlighted herein, while similarities may be omitted. Distal tip 400 may be used in conjunction with medical device 10 in place of distal tip 20.

Distal tip 400 may include a core 402 and a cap 410. Core 402 may be inserted into cap 410 during assembly of distal tip 400. Core 402 may have mounted thereon or, otherwise attached thereto, elements such as imaging device 22 and lighting element 24. In FIGS. 5A-5D, imaging device 22 and lighting element 24 are shown installed/potted. Distal tip 400 may also include an elevator 404, having any of the properties of elevators 26, 104.

Cap 410 of distal tip 400 may extend between a proximal end 412 and a distal end 414. A distal surface 420 may be disposed at distal end 414. Cap 410 may have a first side surface 416 and a second side surface 418. First side surface 416 may be on an opposite side of cap 410 from second side surface 418. First side surface 416 and second side surface 418 may be lateral side surfaces of cap 410. Cap 410 may have a top surface 422 and a bottom surface 424. Top surface 422 may be on an opposite side of cap 410 from bottom surface 424. Top surface 422 of cap 410 may be a side of cap 410 that includes surfaces or openings for receiving electronics (e.g., imaging device 22 and lighting element 24).

Generally, intersections between distal surface 420, first side surface 416, second side surface 418, top surface 422, and bottom surface 424 may be rounded without sharp corners formed at junctions between the various surfaces. Such rounded junctions may provide for smoother interactions between distal tip 400 and surfaces of a subject's body lumen. Distal tip 400 may also have a streamlined shape, such that advancing distal tip 400 requires relatively less force than advancing a conventional distal tip.

First side surface 416, top surface 422, and distal surface 420 may meet at a junction 430. First side surface 416, bottom surface 424, and distal surface 420 may meet at a junction 432. Second side surface 418, bottom surface 424, and distal surface 420 may meet at a junction 434. Second side surface 418, top surface 422, and distal surface 420 may meet at a junction 436.

In contrast to distal tips 100, 200, 300, distal end 414 of distal tip 400 may not be symmetrical. With reference to FIG. 5B (which views distal end 414 looking in a proximal direction), a first side 422a of top surface, which may include imaging device 22 and lighting element 24 (as shown in FIGS. 5C and 5D), may be recessed in a downward direction relative to a second side 422a of top surface 422, which includes an opening 448 for elevator 404. In particular, first side 422a may be further recessed in a downward direction as compared to corresponding structures of distal tips 100, 200, 300. A portion 423 (FIGS. 5C and 5D) of top surface that is distal to imaging device 22 and lighting element 24 may taper upward in a direction toward first side surface 416 (in a laterally outward direction).

Still with reference to FIG. 5B, junctions 430, 432, 434, 436 may be asymmetrical. First side surface 416 may flare outward in an upward direction, such that first side surface 416 may be further from second side surface 418 near top surface 422 than bottom surface 424. Second side surface 418 may be extend in an approximately up/down direction when viewing distal end 414 facing proximally. In contrast, first side surface 416 may slant outwardly in an upward direction, when viewed in the same direction. Junction 436 may fall down to the left in the view of FIG. 5B at a steeper angle than junction 430 falls down to the right.

As shown in FIG. 5A, in particular (a side view facing second side surface 418), top surface 422 may taper gradually downward in a distal direction. A taper of top surface 422 may be generally even, such an intersection between top surface 422 and second side surface 418 forms an approximately straight line. Bottom surface 424 may taper gradually upward. A distal portion 424a of bottom surface 424 may curve upward at a greater rate than more proximal portions of bottom surface 424. An upward angle of distal portion 424a of bottom surface 424 may have a steeper angle than a downward angle o top surface 424.

Referring again to FIG. 5B, radiuses of curvature in the lateral (side to side direction) may be greater for junctions 432 and 434 than for junctions 430 and 436. In other words, a bottom portion of distal end 414/distal surface 420 may be more rounded than a top portion of distal end 414/distal surface 420.

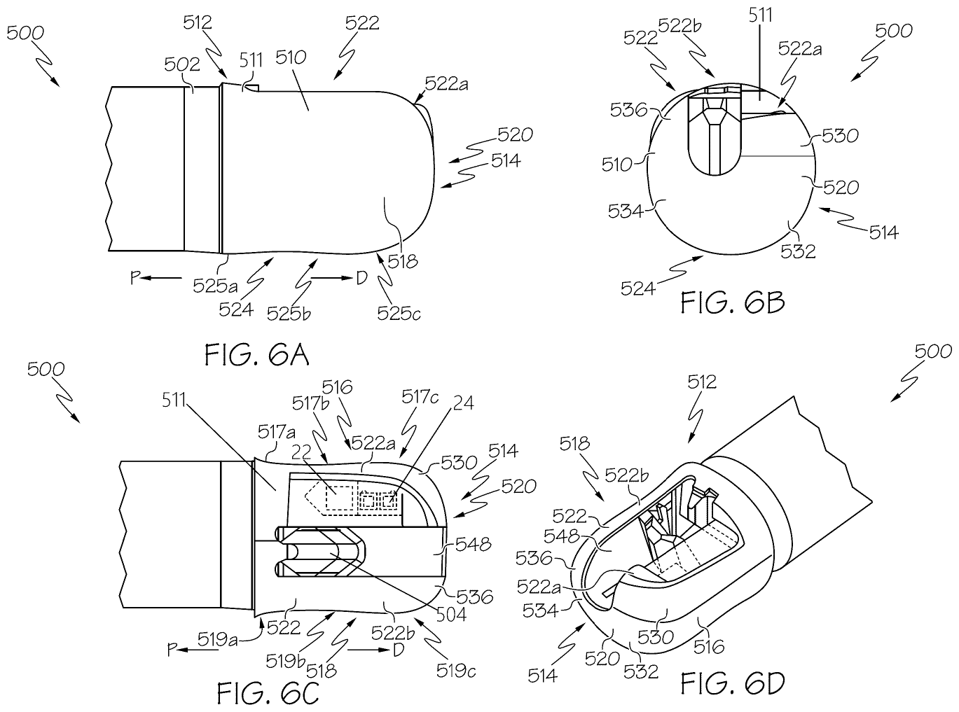

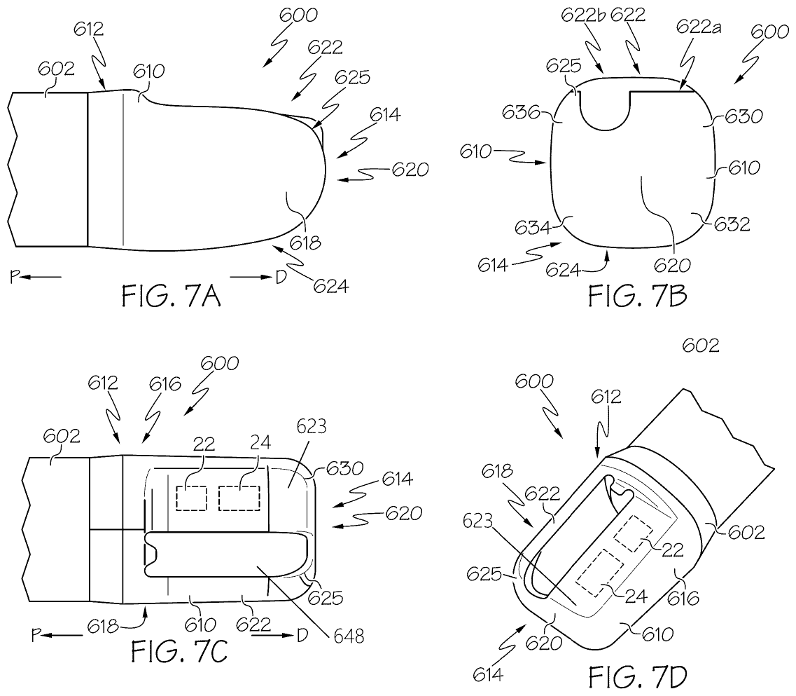

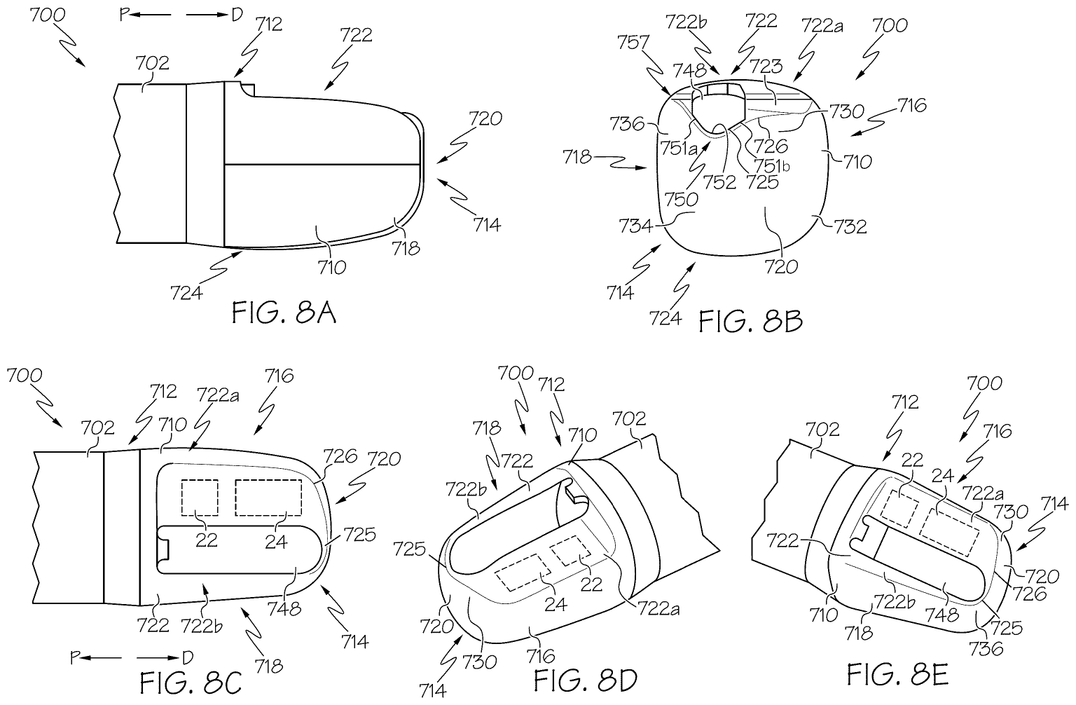

FIGS. 6A-6D depict an alternative distal tip 500, having any of the features of distal tips 100, 200, 300, 400 unless otherwise specified herein. For sake of clarity, differences between distal tip 500 and distal tips 100, 200, 300, 400 may be highlighted herein, while similarities may be omitted. Distal tip 500 may be used in conjunction with medical device 10 in place of distal tip 20.

Distal tip 500 may include a core 502 and a cap 510. Core 502 may be inserted into cap 510 during assembly of distal tip 500. Core 502 may have mounted thereon or, otherwise attached thereto, elements such as imaging device 22 and lighting element 24. Distal tip 500 may also include an elevator 504, having any of the properties of elevators 26, 104, 404.

Cap 510 of distal tip 500 may extend between a proximal end 512 and a distal end 514. A distal surface 520 may be disposed at distal end 514. Cap 510 may have a first side surface 516 and a second side surface 518. First side surface 516 may be on an opposite side of cap 510 from second side surface 518. First side surface 516 and second side surface 518 may be lateral side surfaces of cap 510. Cap 510 may have a top surface 522 and a bottom surface 524. Top surface 522 may be on an opposite side of cap 510 from bottom surface 524. Top surface 522 of cap 510 may be a side of cap 510 that includes surfaces or openings for receiving electronics (e.g., imaging device 22 and lighting element 24).

Generally, intersections between distal surface 520, first side surface 516, second side surface 518, top surface 522, and bottom surface 524 may be rounded without sharp corners formed at junctions between the various surfaces. Such rounded junctions may help provide for smoother interactions between distal tip 500 and surfaces of a subject's body lumen. Distal tip 500 may also have a streamlined shape, such that advancing distal tip 500 (e.g., distally) requires relatively less force than advancing a conventional distal tip.

First side surface 516, top surface 522, and distal surface 520 may meet at a first junction 530. First side surface 516, bottom surface 524, and distal surface 520 may meet at a second junction 532. Second side surface 518, bottom surface 524, and distal surface 520 may meet at a third junction 534. Second side surface 518, top surface 522, and distal surface 520 may meet at a fourth junction 536.

Junctions 530, 532, 534, 536 may have shape similar to junctions 130, 132, 134, 136, when viewing distal end 514 facing a proximal direction, as shown in FIG. 6B. Distal end 514/distal surface 520 of distal tip 500 may have any of the properties of distal end 114/distal surface 120. For example, as shown in FIG. 6B, when viewing distal end 514 in a proximal direction, distal tip 500 may have an approximately circular shape overall. However, similar to distal tip 400, a first portion 522a (having imaging device 22 and lighting element 24) of top surface 522 may be recessed relative to a second portion 522b of top surface 522 that has an opening 548 for receiving elevator 504. Whereas distal surface 120 of distal tip 100 may be symmetrical about various axes (discussed above) and have an overall circular shape when viewed facing proximally (FIG. 2D), proximal portions of side surfaces 516, 518 may contribute to an overall approximately circular shape of distal tip 500 when viewing distal tip 500 in a proximal direction (FIG. 6B). For example, a proximal portion 511 of cap 510 may have an approximately circular shape in a cross-section perpendicular to a central longitudinal axis of distal tip 500.

As shown in FIGS. 6A and 6C, bottom surface 524 and side surfaces 516, 518 may have profiles that first taper inward and then taper outward in a distal direction. Bottom surface 524 may taper upwardly (toward top surface 522), moving distally from a proximal portion 525a to a middle portion 525b of bottom surface 524. From a middle portion 525b to a distal portion 525c of bottom surface 524, bottom surface 524 may taper downwardly moving in a distal direction. In other words, proximal portion 525a and distal portion 525c may be below middle portion 525b. Side surfaces 516, 518 may taper toward one another (laterally inwardly) from proximal portions 517a, 519a, respectively toward middle portions 517b, 519b, respectively, moving in a distal direction. Side surfaces 516, 518 may taper outwardly (laterally outwardly) from middle portions 517b, 519b to distal portions 517c, 519c, moving in a distal direction.

Thus, similarly to distal tip 100, distal tip 500 being wider at distal portions 517c, 519c, 525c than at middle portions 517b, 519b, 525b may help ease movement of distal tip 500 through a body lumen. Middle portions 517b, 519b, 525b may, in at least some circumstances, not make contact with walls of a body lumen, thus decreasing an amount of contact between distal tip 500 and helping make distal tip 500 easier to advance. Proximal portions 517a, 519a, 525a may contact walls of a body lumen. Contact of proximal portions 517a, 519a, 525a my help to prevent middle portions 517b, 519b, 525b from contacting the walls of the body lumen.

As shown in FIGS. 6A and 6D, junctions 530, 532, 534, 536 may be curved when viewing distal tip 500 form the side. A transition between top surface 522 and distal surface 520, as well as between bottom surface 524 and distal surface 520 may have a curve with a large radius of curvature, providing a gradual and smooth transition between the surfaces without sharp edges that may be prone to puncturing tissue.

Top surface 522 may have any of the properties of top surface 422, except as described herein. As compared with other distal tips disclosed herein, top surface 522 may include a larger first portion 522a for receiving imaging device 22 and lighting element 24. As such, top surface 522 may allow for use of larger imaging devices 22 and/or lighting elements 24, which may help improve performance of medical device 10.

FIGS. 7A-7D depict an alternative distal tip 600, having any of the features of distal tips 100, 200, 300, 400, 500, unless otherwise specified herein. For sake of clarity, differences between distal tip 600 and distal tips 100, 200, 300, 400, 500 may be highlighted herein, while similarities may be omitted. In particular, distal tip 600 may share features particularly of distal tips 200, 300. Distal tip 600 may be used in conjunction with medical device 10 in place of distal tip 20.

Distal tip 600 may include a core 602 and a cap 610. Core 602 may be inserted into cap 610 during assembly of distal tip 600. Core 602 may have mounted thereon or, otherwise attached thereto, elements such as imaging device 22 and lighting element 24. Distal tip 600 may also include an elevator (not shown), having any of the properties of elevators 26, 104, 404.

Cap 610 of distal tip 600 may extend between a proximal end 612 and a distal end 614. A distal surface 620 may be disposed at distal end 614. Cap 610 may have a first side surface 616 and a second side surface 618. First side surface 616 may be on an opposite side of cap 610 from second side surface 618. First side surface 616 and second side surface 618 may be lateral side surfaces of cap 610. Cap 610 may have a top surface 622 and a bottom surface 624. Top surface 622 may be on an opposite side of cap 610 from bottom surface 624. Top surface 622 of cap 610 may be a side of cap 610 that includes surfaces or openings (not shown but having any suitable shape or size) for receiving electronics (e.g., imaging device 22 and lighting element 24).

Generally, intersections between distal surface 620, first side surface 616, second side surface 618, top surface 622, and bottom surface 624 may be rounded without sharp corners formed at junctions between the various surfaces. Such rounded junctions may help provide for smoother interactions between distal tip 600 and surfaces of a subject's body lumen. Distal tip 600 may also have a streamlined shape, such that advancing distal tip 600 (e.g., distally) requires relatively less force than advancing a conventional distal tip.

First side surface 616, top surface 622, and distal surface 620 may meet at a first junction 630. First side surface 616, bottom surface 624, and distal surface 620 may meet at a second junction 632. Second side surface 618, bottom surface 624, and distal surface 620 may meet at a third junction 634. Second side surface 618, top surface 622, and distal surface 620 may meet at a fourth junction 636.

An overall shape of distal tip 600 when viewed facing proximally (FIG. 7B) may be similar to distal tips 200 and/or 300, described above. Like distal tip 300, a first portion 622a of top surface 622 (closer to first side surface 616) of distal tip 600 may include distal portion 623, which may be blended with other surfaces of cap 310 and/or have any of the features of portion 323, described above. Unlike distal tip 300, distal tip 600 may lack a cover similar to cover 340. In alternatives, distal tip 600 may include such a cover. Although first portion 622a is shown without an opening for receiving electronics (e.g., imaging device 22 and lighting element 24, labeled in FIGS. 7C and 7D), it will be appreciated that first portion 622a may include such an opening (e.g., as shown for distal tips 400, 500).

A second portion 622b of top surface 622 (closer to second side surface 618) may include an opening 648 for receiving an elevator. Second portion 622b may include a distal portion 625, which may be adjacent to second side surface 618 and distal surface 620. Although distal portion 625 is described as being part of top surface 622, distal portion 625 may alternatively be viewed as a portion of fourth junction 636. Distal portion 625 may taper downward, toward bottom surface 624. Thus, a side profile of second side surface 618/fourth junction 636 (FIG. 7A) may be similar to that of second side surface 118 of distal tip 100 (FIG. 2C). A distal portion of second side surface 618 may be approximately symmetrical about a line extending longitudinally along second side surface 618, halfway between top surface 622 and bottom surface 624.

FIGS. 8A-8E depict an alternative distal tip 700, having any of the features of distal tips 100, 200, 300, 400, 500, 600, unless otherwise specified herein. For sake of clarity, differences between distal tip 600 and distal tips 100, 200, 300, 400, 500, 600 may be highlighted herein, while similarities may be omitted. In particular, distal tip 600 may share features particularly of distal tips 300, 400. Distal tip 700 may be used in conjunction with medical device 10 in place of distal tip 20.

Distal tip 700 may include a core 702 and a cap 710. Core 702 may be inserted into cap 710 during assembly of distal tip 700. Core 702 may have mounted thereon or, otherwise attached thereto, elements such as imaging device 22 and lighting element 24. Distal tip 700 may also include an elevator (not shown), having any of the properties of elevators 26, 104, 404.

Cap 710 of distal tip 700 may extend between a proximal end 712 and a distal end 714. A distal surface 720 may be disposed at distal end 714. Cap 710 may have a first side surface 716 and a second side surface 718. First side surface 716 may be on an opposite side of cap 710 from second side surface 718. First side surface 716 and second side surface 718 may be lateral side surfaces of cap 710. Cap 710 may have a top surface 722 and a bottom surface 724. Top surface 722 may be on an opposite side of cap 710 from bottom surface 724. Top surface 722 of cap 710 may be a side of cap 710 that includes surfaces or openings (not shown but having any suitable shape or size) for receiving electronics (e.g., imaging device 22 and lighting element 24).

Generally, intersections between distal surface 720, first side surface 716, second side surface 718, top surface 722, and bottom surface 724 may be rounded without sharp corners formed at junctions between the various surfaces. Such rounded junctions may help provide for smoother interactions between distal tip 700 and surfaces of a subject's body lumen. Distal tip 700 may also have a streamlined shape, such that advancing distal tip 700 (e.g., distally) requires relatively less force than advancing a conventional distal tip.

First side surface 716, top surface 722, and distal surface 720 may meet at a first junction 730. First side surface 716, bottom surface 724, and distal surface 720 may meet at a second junction 732. Second side surface 718, bottom surface 724, and distal surface 720 may meet at a third junction 734. Second side surface 718, top surface 722, and distal surface 720 may meet at a fourth junction 736.

An overall outer shape/perimeter of distal tip 700 when viewed facing proximally (FIG. 8B) may be similar to distal tip 400, described above. Side surfaces 716, 718 may extend approximately vertically. Bottom surface 724 may slant slightly upwards moving from first side surface 716 to second side surface 718. Thus, second junction 732 may be higher than third junction 734.

A first portion 722a of top surface 722 (closer to first side surface 716) of distal tip 700 may include distal portion 723, which may be blended with other surfaces of cap 710 and have any of the features of portion 323, described above.

A second portion 722b of top surface 722 (closer to second side surface 718) may include an opening 748 for receiving an elevator. As shown particularly in FIG. 8B, second portion 722b may include a distalmost edge 725. Distalmost edge 725 may define a recess 750, with distalmost edge 725 being a rim of recess 750, which may be in communication with opening 748 and may receive an accessory device when the elevator is in a lowered configuration. A distalmost edge 725 of top surface 722 may be disposed around opening 748 and recess 750. Recess 750 (e.g., distalmost edge 725) may have a tapered first portion 751a and a tapered second portion 751b. First portion 751a and second portion 751b may each be straight, curved, or a combination of the both. First portion 751a and second portion 751b may meet at a minimum 752, which may be a concavity. In some examples, first portion 751a and second portion 751b may have a same angle of taper toward minimum 752. In other examples, first portion 751a may be angled more steeply downward than second portion 751b. In examples, second portion 751b may be continuous with a distal edge 726 of portion 723 to form a single, continuous line/curve. Second portion 751b may be shorter than first portion 751a because distalmost edge 725 may be lower than a side 757 of first portion 751a that is opposite minimum 752. An overall shape of distal end 714 may be smooth and blended.

As shown in FIG. 8C, which views distal tip 700 from above, distal surface 720 may be rounded, such that a middle portion of distal surface 720 is further distal than portions of distal surface 720 closer to side surfaces 716, 718. The features of distal end 714 may combine to create a point contact between distal end 714 and tissue, which may help allow for easier gliding or sliding of distal tip 700 through a body lumen.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Additionally, a variety of elements from each of the presented examples can be combined to achieve a same or similar result as one or more of the disclosed embodiments. Thus, the features of the examples should not be viewed as being mutually exclusive or applicable to any particular distal tip and may be combined in any suitable manner. Accordingly, the invention is not to be considered as limited by the foregoing description.

We claim:

1. A distal tip of a duodenoscope, the distal tip comprising:
a first side surface;
a second side surface;
a top surface;
a bottom surface; and
a distal surface, wherein the first side surface, the top surface, and the distal surface meet at a first junction, wherein the first side surface, the bottom surface, and the distal surface meet at a second junction, wherein the second side surface, the bottom surface, and the distal surface meet at a third junction, and wherein the second side surface, the top surface, and the distal surface meet at a fourth junction;
wherein the first junction and the fourth junction are approximately symmetrical, and wherein the second junction and the third junction are approximately symmetrical.

2. The distal tip of claim 1, wherein the first junction and the third junction are approximately symmetrical, and wherein the second junction and the fourth junction are approximately symmetrical.

3. The distal tip of claim 1, wherein, upon viewing the distal surface of the distal tip facing proximally, the distal surface is approximately symmetrical about at least two planes including a line that extends parallel to or coaxial with a central longitudinal axis of the distal tip.

4. The distal tip of claim 3, wherein a portion of the distal surface is approximately symmetrical about at least four planes including a line that extends parallel to or coaxial with a central longitudinal axis of the distal tip.

5. The distal tip of claim 1, wherein the first side surface and the second side surface flare outwardly in a distal direction.

6. The distal tip of claim 1, wherein each of the first side surface and the second side surface includes a proximal portion, a distal portion, and a middle portion, and wherein the middle portion is inward, closer to a central longitudinal axis, of the distal portion and the proximal portion.

7. The distal tip of claim 1, wherein the top surface defines a first opening and a second opening, wherein the first opening is configured to receive an imaging device, and wherein the second opening is configured to receive a lighting element.

8. The distal tip of claim 7, wherein the top surface includes a cover that spans a space between the first opening and the second opening.

9. The distal tip of claim 8, wherein the cover is integrally formed with a remainder of the top surface.

10. The distal tip of claim 1, wherein the top surface includes at least one opening for receiving an imaging device or a lighting element, and wherein a first portion of the top surface that is distal to the at least one opening tapers downward toward the distal surface.

11. The distal tip of claim 10, wherein the first portion curves in a lateral direction.

12. The distal tip of claim 11, wherein the at least one opening is at least one first opening, wherein the top surface further defines a second opening for receiving an elevator, and wherein a side of the first portion that is closer to the second opening is higher than a side of the first portion that is further from the second opening.

13. The distal tip of claim 1, wherein at least a portion of the distal tip has a cross-sectional shape perpendicular to a central longitudinal axis that is approximately circular.

14. The distal tip of claim 1, wherein the top surface tapers downward from a proximal end of the top surface to a distal end of the top surface.

15. The distal tip of claim 1, wherein a central portion of the distal surface extends further in a distal direction than side portions of the distal surface do.

16. A distal tip of a duodenoscope, the distal tip comprising:
a first side surface;
a second side surface;
a top surface;
a bottom surface; and
a distal surface,
wherein each of the first side surface and the second side surface includes a proximal portion, a distal portion, and a middle portion, and wherein the middle portion is inward of the respective distal portion and the respective proximal portion.

17. The distal tip of claim 16, wherein the bottom surface includes a proximal portion, a distal portion, and a middle portion, and wherein the middle portion is higher than the proximal portion and the distal portion.

18. A distal tip of a duodenoscope, the distal tip comprising:
a first side surface;
a second side surface;
a top surface;
a bottom surface; and
a distal surface,
wherein the top surface includes at least one opening for receiving an imaging device or a lighting source, and wherein a first portion of the top surface that is distal to the at least one opening tapers downward toward the distal surface and curves in a lateral direction.

19. The distal tip of claim 18, wherein the at least one opening is at least one first opening, wherein the top surface further defines a second opening for receiving an elevator, and wherein a side of the first portion that is closer to the second opening is higher than a side of the first portion that is further from the second opening.

20. The distal tip of claim 19, wherein a distal portion of the second opening includes a recess defined by a rim, wherein the rim includes an apex and two tapered side portions, and wherein a first of the two tapered side portions is continuous with an edge of the first portion.
